(19) United States
(12) Patent Application Publication
(10) Pub. No.: US 2012/0219634 A1
(43) Pub. Date: Aug. 30, 2012

(54) TOPICAL DERMAL FORMULATIONS

(75) Inventors: John M. Maslowski, Pottstown, PA (US); Declan Daly, Exton, PA (US)

(73) Assignee: Fibrocell Technologies, Inc.

(21) Appl. No.: 13/220,163

(22) Filed: Aug. 29, 2011

Related U.S. Application Data
(60) Provisional application No. 61/377,803, filed on Aug. 27, 2010.
(60) Provisional application No. 61/421,516, filed on Dec. 9, 2010.

(51) Int. Cl. A61K 35/12; C12N 5/0775; A61Q 19/08; A61K 8/98; A61P 17/02

(52) U.S. Cl. 424/572; 435/325

(57) ABSTRACT

An autologous topical formulation containing conditioned medium obtained from culture of autologous fibroblasts has been developed. Unlike other topical formulations, it is autologous since it is derived solely from cells obtained by the person who is to use the formulation. This avoids any possible reaction with proteins derived from the cells. Preferred formulations include gels, creams, lotions, and ointments. The topical formulations of conditioned medium obtained by culturing autologous dermal fibroblasts are topically administered to individuals for the prevention and treatment of scarring, reduction in signs of aging, and improvement in quality of skin.

### CROSS REFERENCE TO RELATED APPLICATIONS

This application claims priority to U.S. Ser. No. 61/377,803 filed Aug. 27, 2010, and U.S. Ser. No. 61/421,516 filed Dec. 9, 2010, both of which are hereby incorporated by reference in their entirety.

### FIELD OF THE INVENTION

This relates to topical formulations for the repair, maintenance and/or long term augmentation of skin in human subjects.

### BACKGROUND OF THE INVENTION

Skin quality deteriorates with age, injury, exposure to sun and other environmental agents, and, on occasion, disease or autoimmune disorders. Over many centuries, a wide variety of materials have been applied, ranging from mud and herbal mixtures, animal fats, to more recent emulsions, lotions, creams, gels, and biologicals. Many of these materials do not contain pharmaceutically active agents, but instead rely on the properties of the relatively inert materials in re-hydrating and soothing the skin. Benefits typically last only as long as the preparation is on the skin.

Many pharmaceutically active agents have been mixed with lotions, gels, creams, solutions, and sprays for topical application. Examples include cortisone and antihistamines to decrease inflammation, antibiotics to treat infection, antifungals, anti-itch, and drying agents. Some include bleaching agents to treat aging spots and chemicals to remove hair. These formulations are very specific to particular active ingredients and do nothing to restore the skin quality or decrease the effects of aging.

More recently, topical biological formulations have been developed. These include formulations containing agents such as cytokines and growth factors, collagen and other extracellular matrix materials, and hydrating compounds such as polyhydroxy acids. Results have been limited in most cases to the specific effect of the biological.

Other biological formulations contain undefined mixtures of active ingredients such as the growth factors, extracellular matrix materials, and other actives found in cell culture media, for example, such as those described by Mehta, et al., in J Drugs Dermatol. September 2008; 7(9):864-71 and in US patent application 20090123503 by Naughton, et al., published May 14, 2009. The problem with many of these formulations, however, is that they are not from the person to whom the mixture is administered and can elicit immune responses to the foreign proteins that can negate the proposed beneficial effects of the culture media.

It is therefore an object of the present invention to provide topical formulations of conditioned medium obtained by culturing autologous dermal fibroblasts for topical administration to patients for the prevention and treatment of scarring, reduction in signs of aging, and improvement in quality of skin.

### SUMMARY OF THE INVENTION

An autologous topical formulation containing conditioned medium obtained from culture of autologous fibroblasts has been developed. Unlike other topical formulations, it is autologous since it is derived solely from cells obtained by the person who is to use the formulation. This avoids any possible reaction with proteins derived from the cells. Preferred formulations include gels, creams, lotions, and ointments.

The topical formulations of conditioned medium obtained by culturing autologous dermal fibroblasts are topically administered to individuals for the prevention and treatment of scarring, reduction in signs of aging, and improvement in quality of skin.

Alternatively, an allogeneic version of the produce may be mass-produced using a similar manufacturing process for use of the general population. In this version of the product, a screened donor would provide tissue for expansion of fibroblasts and creation of a master cell bank (MCB). After appropriate tests are conducted on the MCB, cells expanded from the master bank are used to create a working cell bank (WCB), which is in turn expanded for manufacture of conditioned media for use in the formulation of the allogeneic topical product. The manufacturing process is similar to the autologous process, has the same applications and all final formulations of the topical product will be within the same concentrations ranges.

### DESCRIPTION OF THE DRAWINGS

FIG. 1 is a standardized manufacturing process flow diagram for culturing autologous fibroblasts.

### DETAILED DESCRIPTION OF THE INVENTION

Fibroblasts are specialized cells in the skin that produce collagen and other extracellular matrix components. They are the cells from which connective tissues develop and, as such, play critical roles in the development of human tissue, including the ability to synthesize extracellular matrix components that contribute to skin texture and the secretion of matrix fibers, including collagen. Collagen is a naturally occurring protein that constitutes one of the primary components of the dermis; it exists as a matrix of fibers that provides structure and support. An autologous fibroblast product has been developed. The cell therapy product is composed of a suspension of autologous fibroblasts, grown from a biopsy of each individual's own skin using standard tissue culture procedures. Fibroblasts isolated from the tissue via enzymatic digestion are expanded to a quantity sufficient for injection into the patient's target treatment area. The autologous topical therapy product consists of conditioned culture medium obtained from the expanded fibroblasts, formulated with a suitable excipient for topical administration.

This process may also be used to create allogeneic cell lines that are expanded to create conditioned media for formulation of a mass-produced topical product.

### I. Formulations.

The following definitions are used herein:
ATM Analytical Test Method
AZFICEL-T USAN nomenclature for autologous cultured fibroblasts
BULK HARVEST material following final harvest prior to formulation in cryopreservation media
CGMP Current Good Manufacturing Practice
CS Cell stack
DMEM Dulbecco's Modification of Eagle's Medium
DMSO Dimethyl sulfoxide
DRUG PRODUCT-INJECTION material washed and reformulated in DMEM, vialed and ready for shipment to clinical sites
DRUG SUBSTANCE-CRYOVIAL material formulated in cryopreservation media and aliquoted into cryovials
EDTA Ethylenediaminetetra acetic acid
ES Embryonic System
FACS Fluorescence Activated Cell Sorting
FBS Fetal Bovine Serum
GA Gentamicin and Amphotericin B
IMDM Iscove's Modified Dulbecco's Medium
IND Investigational New Drug application
MCB Master Cell Bank
PBS Phosphate Buffered Saline
PCA Personal Cell Analysis
QC Quality Control
SCNT Somatic Cell Nuclear Transfer
SEC Size Exclusion Chromatography
USP United States Pharmacopeia
WCB Working Cell Bank

A. Conditioned Cell Culture Medium Formulations

A suspension of autologous fibroblasts, grown from a biopsy of each individual's own skin using standard tissue culture procedures, is used to prepare conditioned media for use in a topical autologous formulation. Skin tissue (dermis and epidermis layers) is biopsied from a patient's post-auricular area.

This process may also be used to create allogeneic cell lines that are expanded to create conditioned media for formulation of a mass-produced topical product. The starting material and cellular expansion process to create the Master Cell Bank (MCB) for the allogeneic process is the same as the autologous process.

The fibroblasts can also be used to create other cell types to be cultured to produce materials for use in for tissue repair or regeneration. Derivation of embryonic stem (ES) cells genetically identical to a patient by somatic cell nuclear transfer (SCNT) holds the potential to cure or alleviate the symptoms of many degenerative diseases while circumventing concerns regarding rejection by the host immune system. Byrne, et al., Nature. Nov. 22, 2007; 450(7169):497-502, used a modified SCNT approach to produce rhesus macaque blastocysts from adult skin fibroblasts, and successfully isolated two ES cell lines from these embryos. DNA analysis confirmed that nuclear DNA was identical to donor somatic cells and that mitochondrial DNA originated from oocytes. Both cell lines exhibited normal ES cell morphology, expressed key stem-cell markers, were transcriptionally similar to control ES cells and differentiated into multiple cell types in vitro and in vivo. See also Sparman, et al. Stem Cells. 2009; 27(6):1255-64. FIG. 2 is a schematic from Byrne 2008 Hum. Mol. Gen. 17:R37-R41, showing how skin derived fibroblasts can be de-differentiated using epigenetic reprogramming into pluripotent stem cells, which can then differentiate into neurons, cardiomyocytes, beta islet cells, and hematopoetic cells. See Hochedlinger, et al., Development. February 2009; 136(4):509-23 and Kanawaty, et al. Bioessays. February 2009; 31(2):134-8.

Fibroblasts can be de-differentiated into pluripotent cells by cell fusion (Cowan et al. Science. Aug. 26, 2005; 309(5739):1369-73), direct reprogramming (Takahashi, et al., Cell. 2007 30;131(5):861-72), and somatic cell nuclear transfer (Byrne, et al. 2007). Takahashi, et al. demonstrated the generation of iPS cells from adult human dermal fibroblasts with the same four factors: Oct3/4, Sox2, Klf4, and c-Myc. Human iPS cells were similar to human embryonic stem (ES) cells in morphology, proliferation, surface antigens, gene expression, epigenetic status of pluripotent cell-specific genes, and telomerase activity. Furthermore, these cells could differentiate into cell types of the three germ layers in vitro and in teratomas. These findings demonstrate that iPS cells can be generated from adult human fibroblasts.

B. Preparation of Cells

### Autologous Manufacture:

The autologous fibroblasts are derived by enzymatic digestion of a biopsy of the recipient's own skin followed by expansion in culture using standard cell culture techniques. Skin tissue (dermis and epidermis layers) is biopsied from a subject's post-auricular area. Typically, the starting material is composed of three 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility.

After arrival at the manufacturing facility, the biopsy is inspected and, upon acceptance, transferred directly to the manufacturing area. Upon initiation of the process, the biopsy tissue is washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37±2.0° C. with 5.0±1.0% CO2 and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting.

When cell confluence is greater than or equal to 40% in the T-175 flask, they are trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies. Cells are incubated at 37±2.0° C. with 5.0±1.0% CO₂ and fed every three to five days in the T-500 flask and every five to seven days in the ten layer cell stack (10CS). Cells should not remain in the T-500 flask for more than 10 days prior to passaging. QC release testing for safety of the Bulk Drug Substance includes sterility and endotoxin testing. When cell confluence is ≧95%, cells are passaged to a 10 CS culture vessel, two Five Layer Cell Stacks (5 CS) or a 10 Layer Cell Factory (10 CF). Passage is performed by removing the spent media, washing the cells, and treating with Trypsin-EDTA to release adherent cells in the flasks into the solution. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37±2.0° C. with 5.0±1.0% CO₂ and fed with fresh Complete Growth Media every five to seven days. Cells should not remain in the 10CS for more than 20 days prior to passaging.

Primary Harvest: When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process Quality Control (QC) testing performed to determine total viable cell count and cell viability. The ideal point at which conditioned media should be collected is at this point, since the quantity of media is significantly higher and more cells are present to produce by-products.

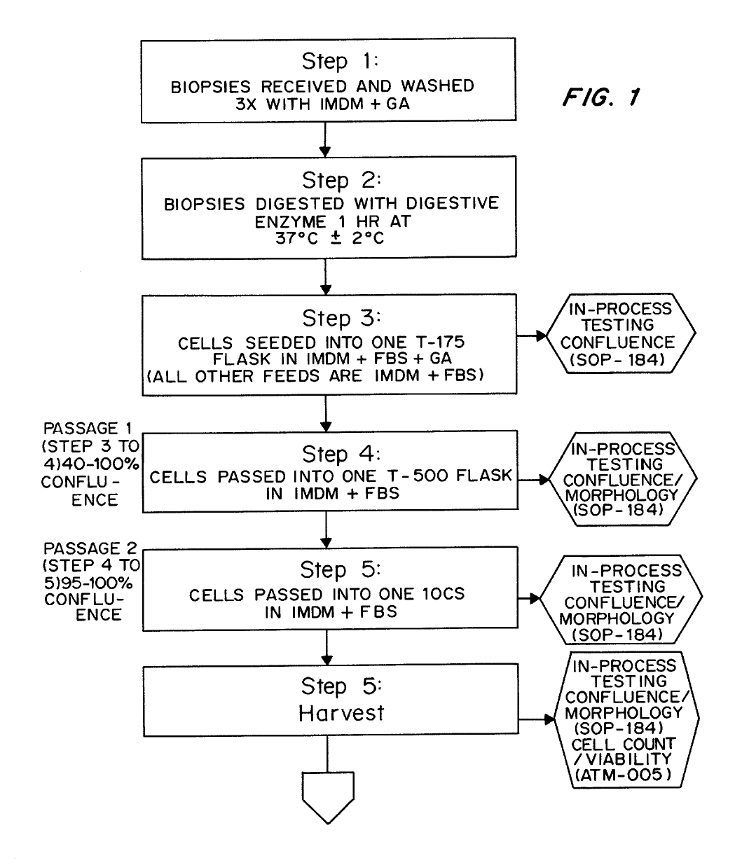

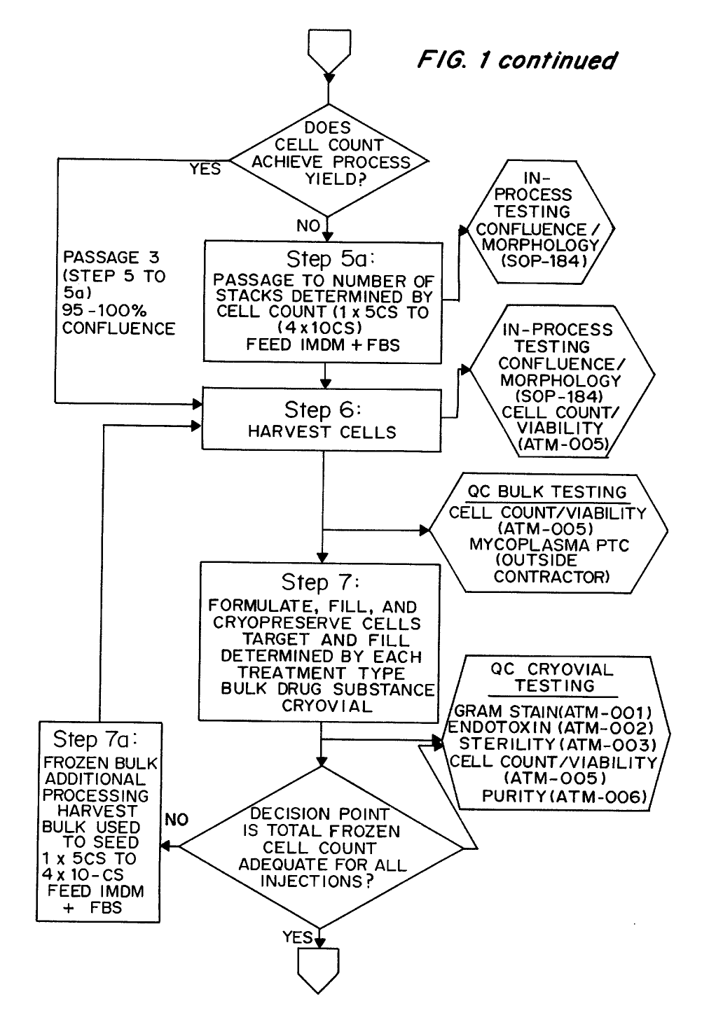

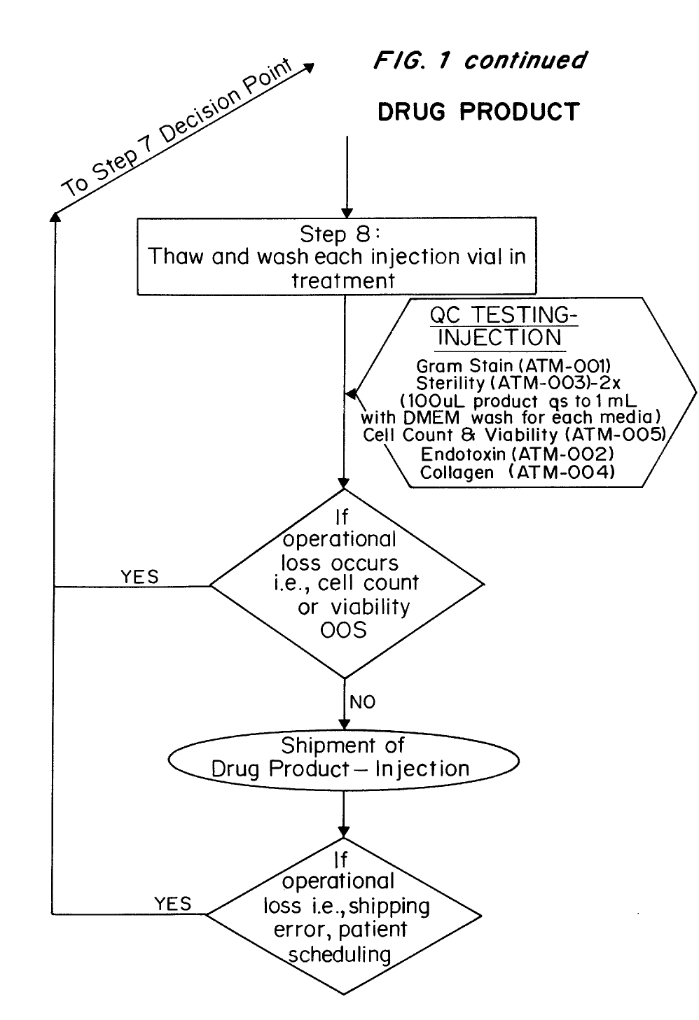

If additional cells are required after receiving cell count results from the primary 10CS harvest, an additional passage into multiple cell stacks (up to four 10CS) is performed (Step 5a in FIG. 1). For additional passaging, cells from the primary harvest are added to a 2 L media bottle containing fresh Complete Growth Media. Resuspended cells are added to multiple cell stacks and incubated at 37±2.0° C. with 5.0±1.0% CO₂ . The cell stacks are fed and harvested as described above, except cell confluence must be 80% or higher prior to cell harvest. The harvest procedure is the same as described for the primary harvest above. A Mycoplasma sample from cells and spent media is collected, and cell count and viability performed as described for the primary harvest above.

Conditioned media for use in preparation of the topical formula is ideally collected from the primary harvest or additional passaging steps after primary harvest from the fibroblast manufacturing process described above. A confluent 10CS consistent of the largest population of adhered cells in comparison to flasks used earlier in the process (T-175 and T-500 flasks) and has the highest volume of Complete Growth Media. However, conditioned media may be collected at any stage of the process where media is removed after a period of incubation to produce a topical product earlier in the process, or pooled for greater yield.

At harvest of the 10CS (or other selected culture vessel), 1.5 L (or less for upstream culture vessels) of conditioned media is removed from the vessel and collected in an appropriate container, such as a 2 L bottle. The media is concentrated by ultrafiltration, dehydration or lyophilization. Concentrated media may be combined from different media harvest points (e.g. T-500 and 10 CS), or individual media harvests may be treated separately.

### Allogeneic Manufacture:

In order to produce the allogeneic fibroblast culture, donors are selected to provide starting tissue. Prior to collection of the biopsy skin tissue, the individual is given a general examination for good health and screened for bloodborne pathogenic diseases, such as HIV and Hepatitis B. Once the donor qualifies for participation, biopsy samples may be collected and shipped as described for the autologous process above.

To provide conditioned media to a large population of customers, a MCB is first established for later cell expansion and media collection. To create the MCB, the biopsies collected from the donor are expanded using the autologous process described above. Once harvest is complete, a series of safety tests may be performed to ensure purity of the cell line, including the following:

Viral screening: Test for a panel of viral particles.

Sterility: Test for the absence of microorganisms.

Mycoplasma: Test specifically for absence of microorganisms classified as Mycoplasma species that are considered a potential contaminant in cell culture.

Endotoxin: Test for proteins causing a pyrogenic (fever) response.

In addition, perform efficacy tests to confirm the quality of the cells:

Cell Count: Quantification of cells in the harvested population.

Cell Viability: Percentage of viable cells in the population.

Identity: Percentage of cells determined to be fibroblasts.

Collagen Content: Amount of collagen present in the cell suspension, indicating a biologically active population of cells.

After final harvest, cells are aliquoted and stored cryogenically in the vapor phase of liquid nitrogen as described for the autologous process above. These cells represent the MCB, and are used downstream to seed additional cultures for conditioned media collection. Maintenance of multiple donor cell lines provide ongoing inventory for manufacturing.

Each MCB vial is capable of seeding a new fibroblast subculture line to create a Working Cell Bank (WCB). Fibroblasts will be passaged in conventional culture vessels to produce enough cells to adequately seed a large scale culture bioreactor. Vials harvested from the culture are frozen and placed into cryostorage to create the finalized WCB.

Frozen cells from the WCB are thawed and expanded using conventional cell culture. Cells are passaged until enough are created to seed a bioreactor. The Bioreactors are commonly used in the biotechnology industry to support the production of vaccines, antibodies and small molecules. To create a large amount of conditioned media for use in formulation of the topical product, a bioreactor may be used to produce a large scale culture to maximize the amount of media collected.

Adherent cell culture in a bioreactor is an existing technology that can be applied directly to this application. A number of off-the-shelf units exist in various sizes that constantly monitor culture conditions such as temperature, CO₂, pH and dissolved oxygen, ensuring consistency from lot to lot. Examples include:

CelliGen® Series (New Brunswick Scientific, Edison, N.J.)

WAVE™ Bioreactor System (GE Healthcare, Piscataway, N.J.)

Bioreactors employ microcarriers to act as the growth surface for adherent cells such as fibroblasts. The carriers are small 2D or 3D structure capable of supporting cell expansion directly to the surface. In large quantities, microcarriers provide a large amount of growth surface area for cells to attach within the bioreactor. Potential carriers include:

Poly blend such as BioNOC II® (Cesco Bioengineering, distributed by Bellco Biotechnology, Vineland, N.J.) and FibraCel® (New Brunswick Scientific, Edison, N.J.)

Gelatin, such as Cultispher-G (Percell Biolytica, Astrop, Sweden)

Cellulose, such as Cytopore™ (GE Healthcare, Piscataway, N.J.) coated/uncoated polystyrene, such as 2D MicroHex™ (Nunc, Weisbaden, Germany), Cytodex® (GE Healthcare, Piscataway, N.J.) or Hy-Q Sphere™ (Thermo Scientific Hyclone, Logan, Utah)

Once seeded into a bioreactor containing microcarriers, the culture is fed with fresh media over the course of the process. Multiple feeds are performed during the culture every few days. The conditioned media is collected aseptically, aliquoted into appropriate containers and frozen for later processing. Alternatively, classic culture vessels such as tissue flaks and Cell Stacks may be used to expand the WCB in place of bioreactors and collect media for use in the topical formulation.

C. Formulations

The carrier may be any gel, ointment, lotion, emulsion, cream, foam, mousse, liquid, spray, suspension, dispersion or aerosol which is capable of delivering actives from the cell culture medium to the tissue. A suitable emulsifying agent is needed if an active agent is insoluble in an aqueous environment. A penetration enhancer may be added to enable the active agents to cross the barrier of the stratum corneum. In one embodiment, the carrier is a gel, which is odorless and tasteless and dissolves rapidly, such as a hydroalcoholic gel.

1. Excipients

“Water Soluble” as used herein refers to substances that have a solubility of greater than or equal to 5 g/100 ml water.

“Lipid Soluble” as used herein refers to substances that have a solubility of greater than or equal to 5 g/100 ml in a hydrophobic liquid such as castor oil.

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase. Excipients for topical administration may include anti-microbial compounds, e.g. parabens, antioxidants, e.g. sodium ascorbyl acetate and alpha-tocopherol, stabilizers, e.g. sorbitol, and/or emulsifying agents to produce a stable emulsion with both a hydrophilic and a hydrophobic phase.

“Diluents” may be included in the formulations to dissolve, disperse or otherwise incorporate the carrier. Examples of diluents include, but are not limited to, water, buffered aqueous solutions, organic hydrophilic diluents, such as monovalent alcohols, and low molecular weight glycols and polyols (e.g. propylene glycol, polypropylene glycol, glycerol, butylene glycol).

Appropriate excipients are selected based on the type of formulation. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars, and starches.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

2. Penetration Enhancers

Penetration enhancers are frequently used to promote transdermal delivery of drugs across the skin, in particular across the stratum corneum. Some penetration enhancers cause dermal irritation, dermal toxicity and dermal allergies. However, the more commonly used ones include urea, (carbonyldiamide), imidurea, N, N-diethylformamide, N-methyl-2-pyrrolidine, 1-dodecal-azacyclopheptane-2-one, calcium thioglycate, 2-pyyrolidine, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.).

3. Formulations

Once the media is sufficiently concentrated, powder or liquid is blended with the selected topical vehicle. Blending can occur manually or using a mechanical device. After formulation, the product is filled into an appropriate dispenser and shipped to the end user. Examples of final container may include a pump bottle, squeeze bottle, jar, tube or vial.

The concentrated material may be either liquid (less than 100 mL) or in powder form depending on the method of concentration used. In test runs, lyophilization of the full 1.5 L collected from the 10CS of fibroblast culture yielded between 23-27 g of powder material (N=3). The concentration conditioned media added is at a range of 1-95% to the excipient depending on intended use, but is typically 1-5%. At these concentrations, multiple container of topical product can be prepared for a single patient for continued supply, or for mass-market sale if soured from allogeneic conditioned media.

4. Conditioned Media Characterization

### Total Collagen:

Testing for total collagen content is part of the release criteria for the injectable autologous cell therapy product, indicating that fibroblasts are biologically active in culture. Conditioned media has also been historically tested for collagen content as part of characterization testing. Testing was conducted using the Sicrol Assay Kit (Biocolor Life Science Assays, United Kingdom). The kit measures collagen I-V and reports a total collagen content value.

Table 1 presents collagen content results from conditioned media taken directly from the culture (N=10, taken from four separate cell culture lots). In addition, the passage number and cell confluence of cells at media collection are presented in Table 1, indicating that collagen is present at various passages in the process at confluency ranging from 40-100% and at different cell passages.

TABLE 1
Collagen content results for conditioned media samples

| | Collagen | | |
|---|---|---|---|
| | Content | % Cell | Cell Culture |
| Sample | (μg/mL) | Confluence | Passage |
| Lot 1 - Sample 1 | 439.34 | 100 | P1 |
| Lot 1 - Sample 2 | 416.90 | 50 | P2 |
| Lot 2 - Sample 1 | 349.36 | 50 | P2 |
| Lot 3 - Sample 1 | 410.74 | 70 | P1 |
| Lot 3 - Sample 2 | 467.72 | 100 | P1 |
| Lot 3 - Sample 3 | 356.84 | 50 | P2 |
| Lot 4 - Sample 1 | 367.74 | 40 | P0 |
| Lot 4 - Sample 2 | 256.74 | 60 | P1 |
| Lot 4 - Sample 3 | 352.44 | 100 | P1 |
| Lot 4 - Sample 4 | 364.32 | 50 | P2 |

### Amino Acids:

The IMDM component of Complete Growth Media contains amino acids in support of cellular expansion. Conditioned media samples collected during the autologous manufacturing process were tested for amino acid content using size exclusion chromatography (SEC, N=6). Table 2 presents the reported concentration ranges for selected amino acids.

TABLE 2
Amino acid content ranges from conditioned media samples

| Amino Acid | Range Detected (mg/L) |
|---|---|
| Asp | 3-17 |
| Glu | 49-105 |
| Ser | 13-33 |
| Asn | 12-16 |
| Gly | 21-48 |
| Gln | 201-282 |
| His | 20-28 |
| Thr | 56-87 |
| Arg | 43-51 |
| Ala | 24-53 |
| Pro | 30-51 |
| Tyr | 41-62 |
| Cys | 19-24 |
| Val | 56-82 |
| Met | 17-25 |
| Ile | 60-85 |
| Leu | 64-93 |
| Lys | 70-102 |
| Phe | 39-60 |

### II. Methods of Administration and Treatment

The preparation may be used for local topical delivery to any location, especially areas in which the skin has thinned, discolored or wrinkled due to age. The aging process of the skin occurs as a result of both intrinsic and extrinsic factors. The factors that contribute to intrinsic or natural aging are both structural and functional. Structurally, the epidermis becomes thinner, the corneocytes are less adherent and the dermal-epidermal junction is flattened. Functionally, there is a reduction in the number and biosynthetic capacity of fibroblasts and the dermis becomes atrophic and relatively acellular and avascular. Exposure to ultraviolet light radiation is the primary cause of extrinsic or photoaging. Extrinsic aging characteristics are loss of elasticity, increased roughness and dryness, irregular pigmentation, deep wrinkling, a leathery appearance, blister formation and impaired wound healing. The visible appearance of aging, especially facial wrinkles and folds, are common effects that patients seek to reduce. Options for the treatment of facial lines, wrinkles and folds include surgery, neurotoxins, fillers, lasers, non-ablative therapies, microdermabrasion and chemical peels. Many of these treatments vary in safety, efficacy, and duration of effect in the treatment of the signs of aging. The formulation described herein may act by stimulating cells in the dermis to grow and divide, by increasing production of extracellular matrix components (e.g. collagen), and/or by stimulating the reorganization of existing extracellular matrix, which may have multifactorial effects for improvement of the skin.

Modifications and variations of the compositions and methods of manufacture and use will be apparent to those skilled in the art from the foregoing detailed description and are intended to come within the scope of the appended claims. All cited references are specifically incorporated by reference.

We claim:

1. A topical formulation comprising
An excipient for topical application, and
Conditioned culture media obtained by culturing biopsied fibroblasts, the fibroblasts being autologous fibroblasts obtained from the intended recipient or the fibroblasts being obtained from one or more individuals screened for disease and compatibility prior to culturing, wherein the excipient is blended with the conditioned culture media or materials therein to form the formulation.

2. The formulation of claim 1 selected from the group consisting of a gel, ointment, lotion, emulsion, cream, foam, mousse, liquid, spray, suspension, dispersion and aerosol.

3. The formulation of claim 1 wherein the fibroblasts have been passaged multiple times to produce the conditioned culture media.

4. The formulation of claim 3 wherein the fibroblasts are passaged after reaching 40% confluence.

5. The formulation of claim 1 wherein the autologous fibroblasts are obtained from three 3-mm punch skin biopsies.

6. The formulation of claim 1 wherein the formulation contains the material obtained from 1.5 liters of conditioned cell culture media where the cells are grown to at least 80% cell confluence.

7. A method of making an autologous topical formulation comprising
An excipient for topical application, and
Conditioned culture media obtained by culturing biopsied fibroblasts, the fibroblasts being autologous fibroblasts obtained from the intended recipient or the fibroblasts being obtained from one or more individuals screened for disease and compatibility prior to culturing,
Comprising culturing the fibroblasts in cell culture, and mixing the conditioned culture media, or materials therein, with the excipient to make a formulation for topical application.

8. The method of claim 7 wherein the formulation is selected from the group consisting of a gel, ointment, lotion, emulsion, cream, foam, mousse, liquid, spray, suspension, dispersion and aerosol.

9. The method of claim 7 wherein the fibroblasts have been passaged multiple times to produce the conditioned culture media.

10. The method of claim 7 wherein the fibroblasts are passaged after reaching 40% confluence.

11. The method of claim 7 wherein the autologous fibroblasts are obtained from three 3-mm punch skin biopsies.

12. The method of claim 7 wherein the formulation contains the material obtained from 1.5 liters of conditioned cell culture media where the cells are grown to at least 80% cell confluence.

13. The method of claim 7 wherein the conditioned cell culture media is dried to produce material which is blended with the excipient.

14. A method for treating skin comprising topically administering formulations of conditioned medium obtained by culturing dermal fibroblasts as defined by claim 1.

15. The method of claim 14 wherein the formulation is administered in an effective amount to reduce scarring or signs of aging.

16. The method of claim 14 wherein the formulation is administered in an effective amount to improve skin quality.
